(19) United States
(12) Patent Application Publication
(10) Pub. No.: US 2024/0374898 A1
(43) Pub. Date: Nov. 14, 2024

(54) SYSTEMS AND METHOD FOR MODULATING THE SPINAL CORD BASED ON SPINAL FIELD POTENTIALS

(71) Applicant: BROWN UNIVERSITY, Providence, RI (US)

(72) Inventors: David Borton, Providence, RI (US); Jonathan S. Calvert, Providence, RI (US); Radu Darie, Providence, RI (US); Samuel Parker, Providence, RI (US)

(21) Appl. No.: 18/562,366

(22) PCT Filed: Jun. 22, 2022
(86) PCT No.: PCT/US2022/034450

§ 371 Date: Nov. 20, 2023

Related U.S. Application Data
(60) Provisional application No. 63/213,842, filed on Jun. 23, 2021.

(51) Int. Cl. A61N 1/36; A61N 1/02; A61N 1/05; A61N 1/378; G16H 20/40

(52) U.S. Cl. CPC A61N 1/36139; A61N 1/025; A61N 1/0551; A61N 1/36062; A61N 1/36146; A61N 1/378; G16H 20/40

(57) ABSTRACT

A system and method are provided to improve physiological function in a patient having a spinal cord lesion or quantify the state of the spinal cord and thus the state of progression of the spinal cord lesion. The system and method use spinal electrophysiological data, such as ECAPs, to determine a patient's physiological state and direct delivery of spinal cord stimulation in response to the determination of the patient's physiological state to achieve a therapeutic effect.

### CROSS-REFERENCE TO RELATED APPLICATIONS

The present application claims priority to U.S. Provisional Application No. 63/213,842 filed on Jun. 23, 2021, which is incorporated by reference herein in its entirety.

### GOVERNMENT SUPPORT CLAUSE

This invention was made with government support under grant number D15AP00112 and D19AC00015 awarded by the Department of Defense.

The government has certain rights in the invention.

### TECHNICAL FIELD

The present disclosure relates to improving physiological function in a patient suffering from a spinal cord injury in response to spinal electrophysiological data.

### BACKGROUND

Spinal cord injury (SCI) is a debilitating condition that disrupts signal propagation and autonomic regulation and has long-term health implications in addition to a heavy financial burden on the individual and society. Following a traumatic SCI, the current standard of care focuses on decompression of the spinal cord injury site, realignment and stabilization of the spinal column, and hemodynamic maintenance to minimize secondary injury. Preliminary results using spinal cord stimulation (SCS) in individuals with SCI have demonstrated restoration of motor function even years post-injury. These demonstrations have included restoration of motor function such as standing, stepping, and voluntary control of muscles below the SCI as well as improvement in bladder and cardiovascular function. However, the primary quantification of the results of the majority of these trials was performed with electromyography (EMG) of the lower extremities in order to evaluate the effect of SCS on motor capacity. Although SCS-evoked EMG responses can provide insight into synaptic events and motor recruitment that occur due to stimulation, the EMG recordings are necessarily indirect and taken from muscles at a large distance from the stimulation site. Therefore, such recordings may not provide the most accurate feedback to program the spinal cord stimulator for SCI rehabilitation.

### SUMMARY

In an aspect, a system is provided to improve or quantify physiological function in a patient having a spinal cord lesion and having a spinal cord stimulator with at least one stimulating electrical contact and at least one sensing electrical contact already implanted or positioned on an epidural surface of the spinal cord. The system can comprise a processor and a non-transitory computer readable medium storing executable instructions executable by the processor to execute the instructions to at least receive sensor data comprising spinal electrophysiological data from the at least one sensing electrical contact; determine the patient's physiological state in response to the sensor data; define neuromodulation parameters for the at least one stimulating electrical contact of the spinal cord stimulator in response to the determination of the patient's physiological state; an direct delivery of a neuromodulation signal via the at least one stimulating electrical contact to the spinal cord based on the neuromodulation parameters to improve the patient's physiological function.

### BRIEF DESCRIPTION OF DRAWINGS

FIG. 1 is a schematic illustration of a spinal cord stimulator positioned on the epidural surface of the spinal cord having a lesion and an implantable pulse generator comprising a processor according to an aspect of the present disclosure.

FIG. 2 is a block diagram of a spinal cord stimulation system according to an aspect of the present disclosure.

FIG. 3 is a block diagram of a spinal cord stimulation system according to an aspect of the present disclosure.

FIG. 4 is a flow chart depicting illustrative steps of a method of improving physiological function in a patient suffering from a SCI according to an aspect of the present disclosure.

### DETAILED DESCRIPTION

As used herein with respect to a described element, the terms “a,” “an,” and “the” include at least one or more of the described elements including combinations thereof unless otherwise indicated. Further, the terms “or” and “and” refer to “and/or” and combinations thereof unless otherwise indicated. The term “machine-learning” can refer to one or more statistical techniques (or algorithms) to progressively improve performance on a specific task without being explicitly programmed. A “predictive model” is a mathematical model or machine learning model that either predicts a future state of a parameter or estimates a current state of a parameter that cannot be directly measured.

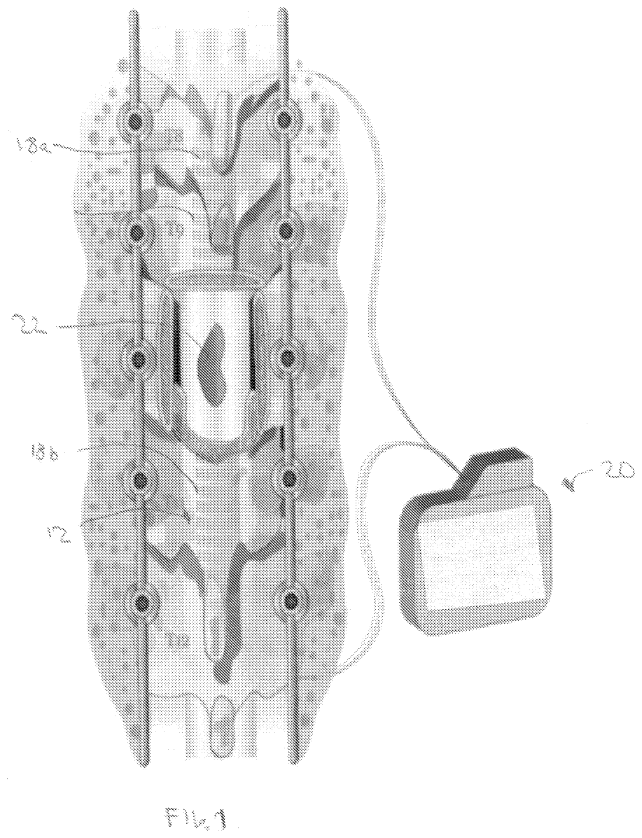

Referring to FIG. 1, in an aspect, a spinal cord stimulation system is provided to improve or quantify physiological function in a patient suffering from a spinal cord lesion including, for example, a spinal tumor, SCI, disc compression on the spinal cord, or combinations thereof. A system can be used to improve function and/or quantify the state of the spinal cord so as to monitor changes/improvements through the use of stimulation and recording of the spinal cord.

The spinal cord stimulator system can include one or more electrical leads 12 that is implantable on the epidural surface of the spinal cord and has at least one stimulating electrical contact 18a and at least one sensing electrical contact 18b. The spinal cord stimulator system can further include a processor, which can be part of an implantable pulse generator (“IPG”) 20 that is in electrical communication with the electrical lead(s). Electrical lead 12 can be a paddle electrode array, a cylindrical lead, or have other configurations. Although FIG. 1 illustrates a stimulating electrical contact rostral to spinal lesion 22 and a sensing electrical contact caudal to spinal lesion 22, the positions could be reversed and the sensing and stimulating electrical contacts can vary in location on the electrode array. Further, the number of stimulating electrical contacts and sensing electrical contacts can vary and can include a plurality of stimulating electrical contacts and a plurality of sensing electrical contacts.

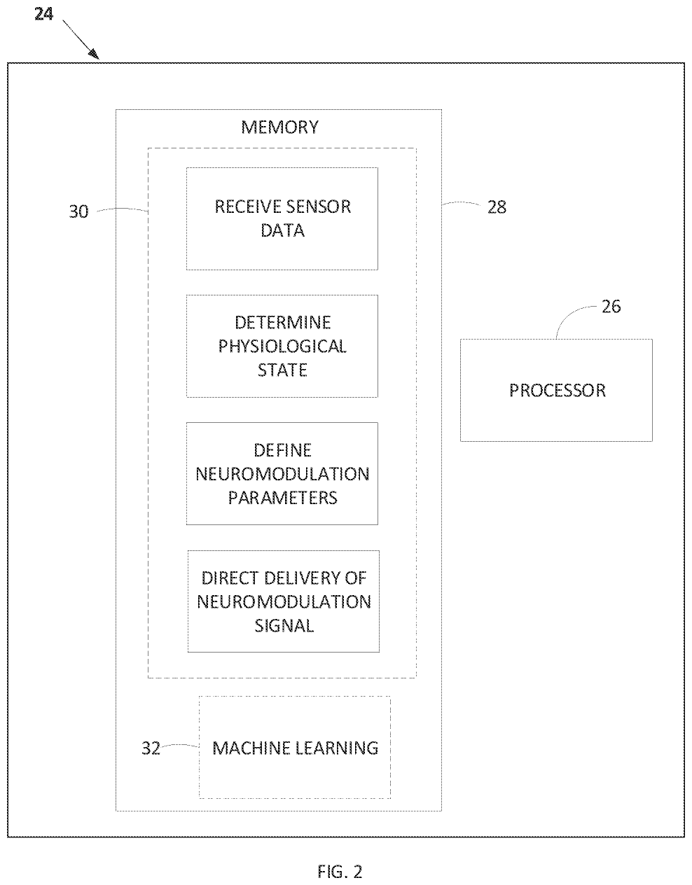

Referring to FIG. 2, in an aspect, a system 24 is provided to improve or quantify physiological function in a patient having a spinal cord lesion and having a spinal cord stimulator with at least one stimulating electrical contact and at least one sensing electrical contact already implanted or positioned on an epidural surface of the spinal cord. System 24 can include processor 26 and non-transitory memory 28 storing computer-readable instructions 30 that, when executed by processor 26, causes the spinal cord stimulator to perform various functions. The computer-readable instructions can include an algorithm for performing the functions and can employ machine learning 32. The instructions can include receiving sensor data comprising spinal electrophysiological data from the at least one sensing electrical contact. The spinal electrophysiological data can include spinal evoked compound action potentials (ECAPs), which are a measure of the response from a population of fibers to an electrical stimulus. The spinal electrophysiological data can also include epidural field potentials (EFPs), which are a measure of the changes in electrical potential caused by spontaneous activity from spinal neurons and axonal fibers. While it does not originate from the spinal cord, an electrocardiogram (EKG) may be extracted from the recordings of epidural voltage. The sensor data can further include EMG data. The instructions can further include analyzing the sensor data to assess or determine the patient's physiological state. For example, the sensor data can be analyzed to determine the patient's bladder fullness, cardiovascular state, visceral state, autonomic state, spinal cord position within the spinal canal, limb position, gait phase or combinations thereof. For example, spectral features (e.g., power in a given frequency band) of the spontaneous electrical activity from a recording electrode could be used to determine bladder fullness or features of muscle activity. Features of the electrical activity rostral to the lesion can be used to determine the patient's intention to void the bladder or intention to move. Neuromodulation that facilitates the activation of these muscles can be delivered to strengthen these spared connections. Furthermore, features of the electrical activity above the lesion may be used to determine the patient's intention to activate said muscles and used to trigger the delivery of neuromodulation. Other examples of a patient's physiological state include the activity of descending efferent fibers of the spinal cord, ascending afferent fibers of the spinal cord, or both. In certain instances, the descending efferent fibers innervate a skeletal muscle and the volitional activation of the skeletal muscle can be assessed by analyzing the sensor data.

Ascending afferent fibers can comprise the ascending afferent fibers of mechanoreceptors, nociceptors, proprioceptors, thermoreceptors, or combinations thereof. Mechanoreceptors are sensory receptors that can detect stimuli such as touch, pressure, vibration, and sound from the external and internal environments. Nociceptors are sensory receptors that can detect signals from damaged tissue or the threat of damage and indirectly also respond to chemicals released from the damaged tissue. Proprioceptors are sensory receptors that can sense the internal forces acting on the body and provide information about the position of the limbs and other body parts in space. Thermoreceptors are sensory receptors that can detect heat and cold and are found throughout the skin in order to allow sensory reception throughout the body. Similarly to the example of determining bladder fullness, features of the spontaneous electrical activity below the lesion can be used to determine, e.g. the angle of a joint. This quantity can be communicated to the patient by varying one dimension of the stimulation delivered above the lesion, e.g. the amplitude at one stimulating electrode.

In addition, in certain aspects one electrical contact of many can be activated, and the evoked response across the rest of the electrical contacts can be recorded. This process can be repeated across all contacts to produce an activation map, where each point has multiple features of the evoked response (time to peaks, distance between peaks, etc.), so, an N(electrode)×M(features)×N(stimulation sites) feature space defines the “state” of the cord. This state can change with limb pose, with injury severity, recovery, etc.

Based on the analysis, neuromodulation parameters can be defined for the at least one stimulating electrical contact of the spinal cord stimulator(s). Non-limiting examples of neuromodulation parameters include electrical contact selection such as which stimulating electrical contact or combination of stimulating electrical contacts should be activated, stimulation patterns, signal pulse waveform, signal pulse width, signal pulse frequency, signal pulse phase, signal pulse polarity, signal pulse amplitude, signal pulse intensity, signal pulse duration, duty cycle, and combinations thereof. The analysis can reveal whether the neuromodulation parameters provide a therapeutic effect. This process can continue until a therapeutic effect is reached. The analyzing can also be aided by machine learning 29. The machine learning 29 can include an algorithm that can be trained to recognize certain physiological states from the recorded sensor data. The machine learning 29 can allow active adjustment of neuromodulation parameters of the spinal cord stimulator. The machine learning can employ one or more machine learning algorithms, such as, for example, Decision tree learning, Association rule learning, Artificial neural networks, Deep learning, Inductive logic programming, Support vector machines, Clustering, Bayesian networks, Reinforcement learning, Representation learning, Similarity and metric learning, Sparse dictionary learning, Genetic algorithms, Rule-based machine learning, Learning classifier systems, Feature selection, or the like.

The instructions can further include providing a predictive model that determines a clinical parameter of spinal cord injury from spinal cord stimulation and the sensor data. The clinical parameter can include, for example, motor outcomes from caudal spinal cord stimulation and EMG and/or spinal electrophysiological recordings, or sensor outcomes from rostral spinal cord stimulation. For instance, following the delivery of a series of diagnostic stimulation pulses from a stimulating electrical contact, a spatiotemporal map of the resulting ECAPs can be collected from the collection of sensing electrical contacts above and below the lesion. The amplitude, dispersion, latency (i.e. timing of peaks w.r.t. to stimulation onset) of the ECAPs can be used to determine connectivity between spinal regions. Neuromodulation can be adjusted to correct for any observed abnormalities, such as left-right asymmetry.

The instructions can further include directing delivery of a neuromodulation signal via the at least one stimulating electrical contact to the spinal cord based on the defined neuromodulation parameters to improve the patient's physiological function. The neuromodulation signal can activate neural activity/neural fibers, block neural activity/neural fibers, or activate neural activity/neural fibers at certain locations of the spinal cord and block neural activity/neural fibers at other locations of the spinal cord.

Regarding specific details of a system as provided herein, the processor can comprise one or more microprocessors under the control of a suitable software program. The processor can control various neuromodulation parameters of the spinal cord stimulator such as, for example, stimulation patterns, electrical contact selection, signal pulse waveform, signal pulse width, signal pulse frequency, signal pulse phase, signal pulse polarity, signal pulse amplitude, signal pulse intensity, signal pulse duration, duty cycle, and combinations thereof. The processor can be programmed to convey a variety of currents and voltages to the stimulating electrical contacts and thereby modulate the activity of a nerve, neuron or nerve fiber in response to recorded spinal electrophysiological data. The processor may be programmed to control numerous electrical contacts independently or in various combinations as needed to provide neuromodulation.

An electrical neuromodulation signal can be constant, intermittent, varying and/or modulated with respect to the current, voltage, pulse width, waveform, duty cycle, frequency, amplitude, and so forth. The waveform can be a sine wave, a square wave, or the like. The type of stimulation may vary and involve different waveforms. Optimal stimulation patterns may require a delay in activating one electrical contact before activating another electrical contact or in another coordinated fashion to improve the patient's physiological function, whether that involves simultaneous activation or staggered activation of electrical contacts in a coordinated, adjustable fashion.

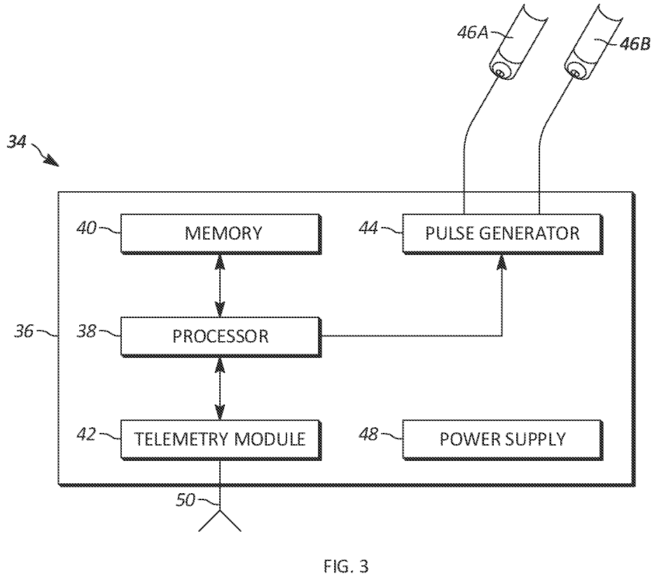

The spinal cord stimulation system can include electronic circuitry, such as one or more electronic circuits for delivering neuromodulation signals enclosed in a sealed housing (such as an IPG) and coupled to spinal cord stimulators, such as microleads. FIG. 3 is a block diagram of a spinal cord stimulation system 34 according to an aspect of the present disclosure. Spinal cord stimulation system 34 can include a housing 36 enclosing a processor 38 and associated memory 40, a telemetry module 42, and a pulse generator 44 in electrical communication with neurostimulator(s) (e.g. electrical leads) 46A and 46B. Spinal cord stimulation system 34 can also include a power supply 48.

Processor 38 can include any one or more of a microprocessor, a digital signal processor (DSP), an application specific integrated circuit (ASIC), a field-programmable gate array (FPGA), or equivalent discrete or integrated logic circuitry. In some examples, processor 38 can include multiple components, such as any combination of one or more microprocessors, one or more processors, one or more DSPs, one or more ASICs, or one or more FPGAs, as well as other discrete or integrated logic circuitry. The functions attributed to processor 38 herein can be embodied as software, firmware, hardware or any combination thereof. In one example, a neurostimulation therapy protocol to improve a patient's physiological function can be stored as instructions in memory 40 that are executed by processor 38 to cause pulse generator 44 to deliver the therapy via neurostimulator(s) 46 according to a protocol based on sensed spinal electrophysiological data.

Memory 40 can include computer-readable instructions that, when executed by processor 38, cause the neurostimulator(s) 46 to perform various functions attributed throughout this disclosure to the neurostimulator(s). The computer-readable instructions can be encoded within memory 40. Memory 40 can comprise non-transitory computer-readable storage media including any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media with the sole exception being a transitory, propagating signal.

Telemetry module 42 and associated antenna 50 can be provided for establishing bidirectional communication with an external device including, for example, a patient programmer and/or a physician programmer. Examples of communication techniques used by spinal cord stimulation system 34 and an external device 18 include low frequency or radiofrequency (RF) telemetry, which can be an RF link established via Bluetooth, WiFi, or MICS, for example. Antenna 50 can be located within, along or extend externally from housing 36.

Power source 48 can include any of a primary battery cell, a rechargeable battery cell, or an inductively coupled power source for providing power for generating and delivering stimulation pulses and powering other device functions such as communication functions. The spinal cord stimulation system (such as the IPG) can include other components such as an analog front end or analog-to-digital converter, a multiplexer and other components.

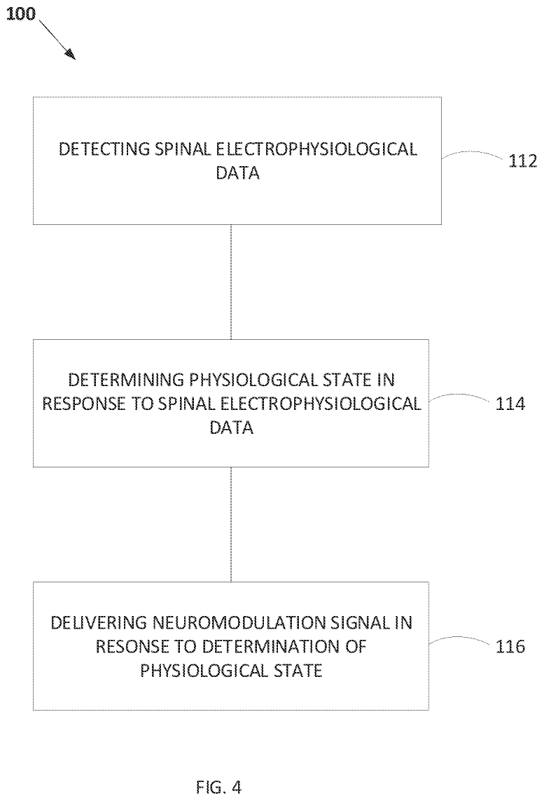

In certain aspects a method of improving physiological function in a patient having a spinal cord lesion is provided. The patient has a spinal cord stimulator with at least one stimulating electrical contact and at least one sensing electrical contact already implanted or positioned on an epidural surface of the spinal cord. Referring to FIG. 4, a method 100 comprises detecting a sensory signal comprising a spinal electrophysiological signal from the at least one sensing electrical contact 112, determining the patient's physiological state in response to the sensory signal 114, and delivering a neuromodulation signal via the at least one stimulating electrical contact to the spinal cord in response to the determination of the patient's physiological state 116. In certain aspects, an initial neuromodulation signal is not delivered to evoke an electrophysiological response but rather the electrophysiological data appears spontaneously and this spontaneous data is analyzed to determine the patient's physiological state. In other aspects, an electrophysiological response is evoked by delivering an initial neuromodulation signal, including a patterned stimulation pattern, and this evoked response is analyzed to determine the patient's physiological state.

With respect to the latter, in certain aspects a method is provided that includes delivering an initial neuromodulation signal having initial stimulation parameters to the patient's spinal cord via the at least one stimulating electrical contact, detecting a sensory signal comprising a spinal electrophysiological signal from the at least one sensing electrical contact in response to the delivery of the initial neuromodulation signal, determining the patient's physiological state in response to the sensory signal, adjusting the initial stimulation parameters in response to the determination of the patient's physiological state, and delivering a subsequent neuromodulation signal with the adjusted stimulation parameters via the at least one stimulating electrical contact to the spinal cord to improve the patient's physiological function. The method can further comprise detecting a subsequent sensory signal comprising a spinal electrophysiological signal from the at least one sensing electrical contact in response to the delivery of the subsequent neuromodulation signal and determining the patient's physiological state in response to the detection of the subsequent sensory signal. This process can continue until a therapeutic effect is reached. In certain aspects, methods can further include generating a spatiotemporal response map based on the spinal electrophysiological data and the patient's physiological state. These spatiotemporal response maps can be evoked via external stimuli or through spontaneous epidural field potentials recorded without external stimuli and can vary across time and space. Further, in any of the methods, the sensory signal can be detected caudal to the level of the spinal cord lesion or rostral to the level of the spinal cord lesion.

Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

What is claimed is:

1. A system to improve or quantify physiological function in a patient having a spinal cord lesion and having a spinal cord stimulator with at least one stimulating electrical contact and at least one sensing electrical contact already implanted or positioned on an epidural surface of the spinal cord, the system comprising:
a processor, and
a non-transitory computer readable medium storing executable instructions executable by the processor to execute the instructions to at least:
receive sensor data comprising spinal electrophysiological data from the at least one sensing electrical contact;
determine the patient's physiological state in response to the sensor data;
define neuromodulation parameters for the at least one stimulating electrical contact of the spinal cord stimulator in response to the determination of the patient's physiological state; and
direct delivery of a neuromodulation signal via at least one stimulating electrical contact to the spinal cord based on the neuromodulation parameters to improve the patient's physiological function.

2. The system of claim 1, wherein the spinal electrophysiological data comprises evoked spinal compound action potentials (ECAPs).

3. The system of claim 1, wherein the patient's physiological state comprises bladder fullness, cardiovascular state, visceral state, autonomic state, spinal cord position within a spinal canal, limb position, gait phase, or combinations thereof.

4. The system of claim 1, wherein the patient's physiological state comprises the activity of ascending afferent fibers, descending efferent fibers, or both.

5. The system of claim 4, wherein the ascending afferent fibers comprise the ascending afferent fibers of mechanoreceptors, nociceptors, proprioceptors, thermoreceptors or combinations thereof.

6. The system of claim 1, wherein the descending efferent fibers innervate a skeletal muscle and the patient's physiological state comprises the volitional activation of the skeletal muscle.

7. The system of claim 1, wherein the sensor data comprises EMG data.

8. The system of claim 1, wherein the neuromodulation parameters are defined by applying a trained machine learning algorithm to the sensor data.

9. The system of claim 1, further comprising instructions to provide a predictive model that determines a clinical parameter representing spinal cord injury from the sensor data and the delivery of the neuromodulation signal.

10. A method of improving physiological function in a patient having a spinal cord lesion and having a spinal cord stimulator with at least one stimulating electrical contact and at least one sensing electrical contact already implanted or positioned on an epidural surface of the spinal cord, the method comprising:
detecting sensor data comprising spinal electrophysiological data from the at least one sensing electrical contact;
determining the patient's physiological state in response to the sensory data; and
delivering a neuromodulation signal via the at least one stimulating electrical contact to the spinal cord in response to the determination of the patient's physiological state to improve the patient's physiological function.

11. A method of improving physiological function in a patient having a spinal cord lesion and having a spinal cord stimulator with at least one stimulating electrical contact and at least one sensing electrical contact already implanted or positioned on an epidural surface of the spinal cord, the method comprising:
delivering an initial neuromodulation signal having initial stimulation parameters to the patient's spinal cord via the at least one stimulating electrical contact;
detecting sensor data comprising spinal electrophysiological data from the at least one sensing electrical contact in response to the delivery of the initial neuromodulation signal;
determining the patient's physiological state in response to the sensor data;
adjusting the initial stimulation parameters in response to the determination of the patient's physiological state; and
delivering a subsequent neuromodulation signal with the adjusted stimulation parameters via the at least one stimulating electrical contact to the spinal cord to improve the patient's physiological function.

12. The method of claim 11, further comprising:
detecting subsequent sensor data comprising spinal electrophysiological data from via the at least one sensing electrical contact in response to the delivery of the subsequent neuromodulation signal; and
determining the patient's physiological state in response to the detection of the subsequent sensor data.

13. The method of claim 11, further comprising generating a spatiotemporal map of activation of the spinal cord based on delivery of the initial neuromodulation signal, the detection of the sensor data, delivery of the subsequent neuromodulation signal, and the detection of the subsequent sensor data.

14. The method of claim 10, wherein detecting comprises detecting sensor data caudal to the level of the spinal cord lesion.

15. The method of claim 10, wherein detecting comprises detecting a sensor data rostral to the level of the spinal cord lesion.
